(19) United States
(12) Patent Application Publication
(10) Pub. No.: US 2012/0203032 A1
(43) Pub. Date: Aug. 9, 2012

(54) PROCESS FOR PRODUCING AND PURIFYING A CONCENTRATED LACTIC SALT

(75) Inventors: Aurelie Bernard, Tournal (BE); Nicolas Delmotte, Kain (BE); Jean-Christophe Bogaert, Ligne (BE); Pierre-Antoine Mariage, Pipaix (BE)

(73) Assignee: GALACTIC S.A., Escanaffles (BE)

(21) Appl. No.: 13/364,775

(22) Filed: Feb. 2, 2012

(30) Foreign Application Priority Data
Feb. 3, 2011 (BE) 2011/0066

(51) Int. Cl. C07C 59/08; C07C 51/43; C07C 51/347

(52) U.S. Cl. 562/580; 562/589

(57) ABSTRACT

The present disclosure relates to a process for preparing a lactic salt from a lactide starting material. In particular, the present disclosure is directed to the production of potassium lactate, wherein the potassium lactate has a desirable neutral odor and a concentration greater than 75%, and wherein the potassium lactate possesses desirable properties for food applications.

### FIELD OF THE INVENTION

The present disclosure relates to a process for producing and purifying a highly concentrated lactic salt obtained from a lactide starting material. In particular, the present disclosure relates to a process for producing a highly concentrated potassium lactate, and sufficiently purifying the potassium lactate to be odorless. In particular, the process of the present disclosure may be advantageously conducted using a lower power consumption compared to processes known in the art.

### STATE OF THE ART

Lactic salts, for example sodium lactate and potassium lactate, are used in the food processing industry. The lactic salts are mainly used in the meat products industry to limit bacterial growth and thus extend the shelf-life of products. The potentially negative impact of sodium on health implies an obligation to reduce the content of sodium, including sodium lactate, in food products. For example, the use of potassium lactate instead of sodium lactate allows the sodium concentration to be reduced in food, while preserving an acceptable preservation efficiency over time.

Thus, there is a need in the market for a lactic salt, such as potassium lactate, with a concentration of greater than 60% (for example, greater than 65%, 75%, or 80%). In particular, a lactic salt with a higher concentration could potentially reduce transport costs, reduce the amount of additional water added to food dyes, and reduce the power consumption required for production.

For example, due to reasons of stoichiometric and material balance, the synthesis of potassium lactate from 100% lactic acid and potassium hydroxide as a liquid at room temperature does not allow for formation of potassium lactate with a concentration greater than 67%. To that end, European Patent No. EP1797773 teaches a process for obtaining a highly concentrated potassium lactate solution by concentrating potassium lactate from 65% to 80% via evaporation. However, the evaporation step according to EP1797773 requires a high expenditure of power. The expenditure of power according to EP1797773 is assessed at about 810 kJ/kg of product, and undesirably requires expensive evaporation facilities.

It has been surprisingly discovered that the synthesis of a highly concentrated lactic salt can be obtained by starting with a lactic acid cyclic ester such as 3,6-dimethyl-1,4-dioxane-2,5-dione (i.e., lactide or dilactide). However, products obtained from starting materials such as a lactide typically have a detectable and clearly identifiable odor, thus making the product undesirable for the food industry.

The state of the art makes use of several treatments for reducing or even removing the undesirable odor of lactic salt solutions, in particular potassium lactate. For example, Great Britain Patent No. GB441453 teaches an oxidation treatment of lactides using either ozone, or hydrogen peroxide. In turn, the process described in WO02/090311 provides a deodorization treatment of potassium lactate by passage through activated carbons. However, it has been observed that potassium lactate produced from a lactide starting material preserves the undesirable odor, in spite of resorting to the varying techniques described in the art.

Thus, a need exists for a process of producing a lactic salt (such as a highly concentrated potassium lactate) from a lactide starting material wherein the lactic salt possesses desirable properties required for the food industry, and wherein the lactic salt advantageously requires a lower power consumption for production.

Accordingly, the present disclosure describes a process for obtaining a lactic salt with a concentration greater than 60% and with a desirable, neutral odor. Furthermore, the present disclosure describes that the process may be advantageously performed at an energy cost (e.g., approximately 152 kJ/kg of product) that is much lower compared to the processes described in the art. In addition, the present disclosure further describes a process for obtaining a lactic salt comprising a purification step of promoting growth of lactide degradation products that are responsible for the undesirable odor, and then removing the lactide degradation products to result in a lactic salt with a desirable, neutral odor.

Furthermore, the present disclosure describes a process for obtaining a lactic salt from lactide in which the lactic salt has properties required for a food application. In particular, the lactic salt may have a coloration lower than 50 Hazen and, thus, will not have the undesirable odor.

The lactic salt solution color is often expressed in Hazen units, in accordance with the APHA/Hazen/Platinium-Cobalt color scale. The three designations of the APHA/Hazen/Platinium-Cobalt color scale are generally used in different application areas but are based on standardized procedures resorting to the same measurement principle: ASTM D 1209 “Standard Method for Color of Clear Liquids (Platinum-Cobalt Scale),” BS 5339:76 (1993) “Measurement of Color on Hazen Units (Platinum-Cobalt Scale),” DIN 53409 “Bestimmung der Hazen-Farbzahl (APHA-Verfahren),” and DIN ISO 6271 “Einstufung der Farbe nach der Platin Cobalt Skala.”

### BRIEF DESCRIPTION OF THE INVENTION

The present disclosure relates to a process for preparing a lactic salt from a lactide starting material, wherein the lactic salt has a desirable neutral odor and a concentration greater than 75%, and wherein the lactic salt possesses desirable properties for food applications. In particular, the present disclosure is directed to the production of potassium lactate according to the described process.

### BRIEF DESCRIPTION OF THE DRAWINGS

FIG. 1 shows a summary of the percentage of people of a 25-person sensory panel that detected an odor as a function of potassium lactate concentration. A potassium lactate solution prepared without heat treatment is compared to a potassium lactate solution prepared with heat treatment.

FIG. 2 shows a summary of the number of people of a 25-person sensory panel according to the type of detected odor (e.g., neutral, acidic, fermented, or lactide).

### DETAILED DESCRIPTION OF EMBODIMENTS OF THE INVENTION

It has been discovered a process for producing and purifying a monovalent or divalent lactic salt with a concentration substantially greater than 60%, wherein the lactic salt has a desirable, neutral odor and is prepared using a low energy cost.

In particular, the present disclosure describes an energetically efficient process for producing a potassium lactate solution with a coloration value lower than 50 Hazen, a desirable neutral odor, and a concentration greater than 75%.

In one embodiment described herein, a process for producing a lactic salt wherein the lactic salt has a concentration greater than 60% is provided. The process comprises the following steps:

A) combining a lactide and an alkaline compound to form a reaction mixture;

B) heating the reaction mixture to a temperature between about 115° C. and about 160° C. for a duration of time between about 2 to about 6 hours, wherein the heating promotes the growth of an odorant or an odorant precursor compound; and

C) purifying the product of step B) to remove the odorant or the odorant precursor compound.

In various embodiments, the combining of the reaction mixture is performed when the lactide is molten. In other embodiments, the combining of the reaction mixture is performed when the alkaline compound is in a solution.

In some embodiments, the lactic salt is a monovalent salt. In some embodiments, the monovalent salt is selected from the group consisting of potassium lactate and sodium lactate. In one embodiment, the monovalent salt is potassium lactate. In another embodiment, the monovalent salt is sodium lactate.

In some embodiments, the lactic salt is a divalent salt. In some embodiments, the divalent salt is selected from the group consisting of calcium lactate and magnesium lactate. In one embodiment, the divalent salt is calcium lactate. In another embodiment, the divalent salt is magnesium lactate.

In some embodiments, the lactic salt produced according to the process has a concentration greater than 60%. In other embodiments, the lactic salt produced according to the process has a concentration greater than 65%. In other embodiments, the lactic salt produced according to the process has a concentration greater than 70%. In other embodiments, the lactic salt produced according to the process has a concentration greater than 75%. In other embodiments, the lactic salt produced according to the process has a concentration greater than 80%. In yet other embodiments, the lactic salt produced according to the process has a concentration greater than 85%. In other embodiments, the lactic salt produced according to the process has a concentration greater than 90%.

In some embodiments, the alkaline is selected from the group consisting of an alkaline oxide, an alkaline hydroxide, an alkaline carbonate, an alkaline bicarbonate, and an alkaline earth metal. In one embodiment, the alkaline is sodium hydroxide. In another embodiment, the alkaline is potassium hydroxide. In another embodiment, the alkaline is calcium carbonate. In yet another embodiment, the alkaline is magnesium hydroxide.

According to various embodiments, the step of combining of the lactide and the alkaline compound to form a reaction mixture is performed with a catalyst. In other embodiments, the step of combining of the lactide and the alkaline compound to form a reaction mixture is performed without a catalyst.

According to some embodiments, the step of heating of the reaction mixture is performed at a temperature between about 130° C. and about 140° C. In other embodiments, the step of heating of the reaction mixture is performed for a duration of time between about 3 to about 4 hours.

According to various embodiments, the step of heating of the reaction mixture is performed under reflux. In other embodiments, the step of heating of the reaction mixture is performed without reflux.

According to some embodiments, the step of heating of the reaction mixture is performed using pressure. In other embodiments, the step of heating of the reaction mixture is performed without using pressure.

According to various embodiments, the step of purifying the product to remove the odorant or the odorant precursor compound is performed using an activated carbon bed.

According to other various embodiments, the purified product subsequently undergoes crystallization, evaporation, or drying.

According to some embodiments, the process further comprises step D) recovering the purified product in which the odorant or the odorant precursor compound has been removed.

In another embodiment of the present disclosure, a purified lactic acid is provided. In various embodiments, the purified lactic acid is prepared according to the described process. The previously described embodiments of the process for producing a lactic salt are applicable to the purified lactic acid described herein.

In some embodiments, the purified lactic acid has a neutral odor. In other embodiments, the purified lactic acid has a coloration lower than about 50 Hazen. In one embodiment, the purified lactic acid has a coloration lower than about 40 Hazen. In one embodiment, the purified lactic acid has a coloration lower than about 30 Hazen. In one embodiment, the purified lactic acid has a coloration lower than about 20 Hazen. In one embodiment, the purified lactic acid has a coloration lower than about 10 Hazen.

In yet other embodiments, the purified lactic acid is prepared using an energy of less than about 800 kJ per kg of product. In one embodiment, the purified lactic acid is prepared using an energy of less than about 700 kJ per kg of product.

In one embodiment, the purified lactic acid is prepared using an energy of less than about 600 kJ per kg of product. In one embodiment, the purified lactic acid is prepared using an energy of less than about 500 kJ per kg of product. In one embodiment, the purified lactic acid is prepared using an energy of less than about 400 kJ per kg of product. In one embodiment, the purified lactic acid is prepared using an energy of less than about 300 kJ per kg of product. In one embodiment, the purified lactic acid is prepared using an energy of less than about 200 kJ per kg of product. In one embodiment, the purified lactic acid is prepared using an energy of about 150 kJ per kg of product.

Further details and features of the invention, given hereinafter by way of non-limiting examples, will become clearer from the description as well as possible embodiments.

### Example 1

### Exemplary Process Using Potassium Hydroxide

According to one embodiment of the present disclosure, the reaction of molten lactide with potassium hydroxide may be carried out according to the following conditions.

A. Reaction Between Lactide and a Lactic Salt Solution:

The first step of the process comprises reacting molten lactide with a potassium hydroxide solution with a concentration greater than 57%, preferably greater than 63%, that has previously been heated to about 70° C. At this concentration, potassium hydroxide crystallizes at room temperature. Potassium hydroxide may be gradually added to the molten lactide in order to control the pH of the solution to approximately 11. After the reaction, the pH of the solution is decreased to a value of approximately 8.

B. Growth of Lactide Degradation Compounds:

In some embodiments of the present disclosure, lactide degradation compounds can be subsequently removed by heating the potassium lactate formed under reflux at a temperature between approximately 115 to 160° C. for a duration of about 2 to 6 hours. In one embodiment, the potassium lactate is heated at a temperature between approximately 135 and 140° C. In one embodiment, the potassium lactate is heated for a duration of about 3 to 4 hours. This step allows for the promotion of forming an odorant or an odorant precursor compound due to the degradation of lactide, thus making their subsequent removal easier. Alternatively, in another embodiment, the continuously formed condensates may be removed while compensating the volume of the solution by adding water in order to maintain a constant product concentration. This treatment has the advantage of continuously removing the volatile derivatives that may be released during the heat treatment (e.g., ethanol, acetate, acetaldehyde, glyceraldehyde, etc.).

In another embodiment, the heat treatment of the potassium lactate is performed under pressure in order to achieve a higher temperature. According to this embodiment, the treatment is performed more quickly, while considering that the risk of heat degradation of potassium lactate itself strongly increases above 170° C.

C. Purification of Highly Concentrated Potassium Lactate:

Highly concentrated potassium lactate may then be purified in order to remove the lactide degradation products by any technique known to those skilled in the art. For example, the purification step may be performed via passage of the material through an activated carbon bed.

According to the present disclosure, the molten lactide can be reacted with a calcium or magnesium oxide or hydroxide and then the mixture can be subjected to the described process prior to performing crystallization, evaporation, or drying of the formed divalent lactic salt. The reaction may be performed using a technique known to those skilled in the art (e.g., a fluidized bed, atomization, etc.).

### Example 2

### Process without Heat Treatment

Approximately 1 kg of lactide can be molten at 105° C. in a reactor. A KOH solution at a concentration of approximately 65% can be preheated to about 70° C. After the lactide is molten, approximately 1215 g of the preheated KOH can be added to the molten lactide at a flow rate over approximately 4 hours. The pH can then be increased to approximately 11 and the temperature can be maintained at about 115° C. Subsequently, the pH can be lowered to a value of approximately 8 with 95% lactic acid.

The potassium lactate (i.e., “PL”) thus obtained can then be passed through a 200 ml Norit ROX-type activated carbons column at 0.2 BV/h (with “By” being defined as one volume equivalent to the activated carbon volume contained in the treatment column). Table 1 summarizes the characteristics of the potassium lactate produced according to this process.

TABLE 1
Characteristics of potassium lactate after passage through activated
carbons

| Coloration (Hazen) | PL content (%) |
|---|---|
| 10 | 80.2 |

### Example 3

### Process with Heat Treatment

Approximately 1 kg of lactide can be molten at 105° C. in a reactor. A KOH solution at a concentration of approximately 65% can be preheated to about 70° C. After the lactide is molten, approximately 1215 g of the preheated KOH can be added to the molten lactide at a flow rate over approximately 4 hours. The pH can then be increased to approximately 11 and the temperature can be maintained at about 115° C. Subsequently, the pH can be lowered to a value of approximately 8 with 95% lactic acid.

The potassium lactate thus obtained can then be heated under reflux at a temperature of approximately 135° C. for about 4 hours in order to promote growth of lactide degradation products.

The solution can then be passed by percolation through a 200 ml Norit ROX-type activated carbons column at 0.2 BV/h. Table 2 summarizes the characteristics of the potassium lactate produced according to this process.

TABLE 2
Characteristics of potassium lactate of the invention

| Coloration (Hazen) | PL content (%) |
|---|---|
| 8 | 80.3 |

### Example 3

### Organoleptic Comparison of the Two Highly Concentrated Potassium Lactates

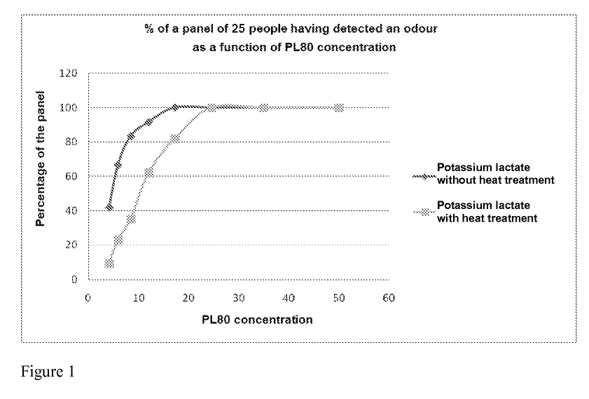

The two potassium lactate solutions obtained according to Examples 2 and 3 can be analyzed by a sensory panel of 25 people in order to determine the detection limit of the potassium lactate odor as a function of concentration. Table 3 summarizes the percentage of people on the sensory panel that detected an odor as a function of potassium lactate concentration. Table 3 shows that the PL80 obtained according to the process of Example 3 has a more desirable odor compared to the PL80 obtained according to the process of Example 2. The results are also displayed in FIG. 1.

TABLE 3
Percentage of a panel of 25 persons having detected an
odor as a function of the potassium lactate concentration

| | PL80 Concentration (%) | | | | | | | |
|---|---|---|---|---|---|---|---|---|
| | 4.1 | 5.9 | 8.4 | 12 | 17.2 | 24.5 | 35 | 50 |
| Percentage | 41.67 | 66.67 | 83.33 | 91.67 | 100 | 100 | 100 | 100 |
| of the panel | | | | | | | | |
| detecting the | | | | | | | | |
| odor of | | | | | | | | |
| potassium | | | | | | | | |
| lactate | | | | | | | | |
| formed | | | | | | | | |
| according to | | | | | | | | |
| Example 2 | | | | | | | | |
| Percentage | 9.09 | 23 | 35 | 62 | 81.82 | 100 | 100 | 100 |
| of the panel | | | | | | | | |
| detecting the | | | | | | | | |
| odor of | | | | | | | | |
| potassium | | | | | | | | |
| lactate | | | | | | | | |
| formed | | | | | | | | |
| according to | | | | | | | | |
| Example 3 | | | | | | | | |

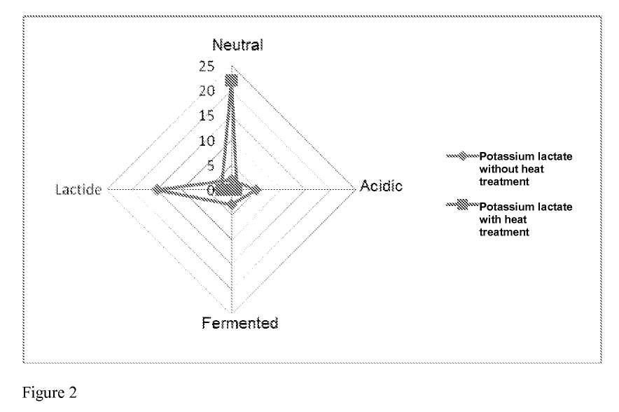

During the organoleptic test, each person of the panel also assessed the type of detected odor by selecting from the following odorant characteristics: neutral, acidic, fermented and lactide. Table 4 summarizes the odorant characteristics described. The results are also displayed in FIG. 2.

TABLE 4
Description of odorant characteristics of highly concentrated
potassium lactates

| | Number of persons | | | |
|---|---|---|---|---|
| | Neutral | Acidic | Fermented | Lactide |
| | odor | odor | odor | odor |
| Potassium lactate | 2 | 5 | 3 | 15 |
| without heat treatment | | | | |
| Potassium lactate of the | 22 | 1 | 0 | 2 |
| invention | | | | |

The potassium lactate formed without heat treatment (i.e., the potassium lactate formed according to Example 2) was mainly described as having a lactide characteristic odor. In comparison, potassium lactate formed with heat treatment (i.e., the potassium lactate formed according to Example 3) was mainly described as having a neutral odor.

While the invention has been illustrated and described in detail in the foregoing description, such an illustration and description is to be considered as exemplary and not restrictive in character, it being understood that only the illustrative embodiments have been described and that all changes and modifications that come within the scope of the invention are desired to be protected. Those of ordinary skill in the art may readily devise their own implementations that incorporate one or more of the features described herein, and thus fall within the scope of the present invention.

1. A process for producing a lactic salt wherein the lactic salt has a concentration greater than 60%, the process comprising the steps of:
A) combining a lactide and an alkaline compound to form a reaction mixture;
B) heating the reaction mixture to a temperature between about 115° C. and about 160° C. for a duration of time between about 2 to about 6 hours, wherein the heating promotes the growth of an odorant or an odorant precursor compound; and
C) purifying the product of step B) to remove the odorant or the odorant precursor compound.

2. The process of claim 1 wherein the lactic salt is a monovalent salt.

3. The process of claim 2 wherein the monovalent salt is potassium lactate.

4. The process of claim 1 wherein the lactic salt is a divalent salt.

5. The process of claim 1 wherein the lactic salt has a concentration greater than 75%.

6. The process of claim 1 wherein the alkaline is selected from the group consisting of an alkaline oxide, an alkaline hydroxide, an alkaline carbonate, an alkaline bicarbonate, and an alkaline earth metal.

7. The process of claim 1 wherein the alkaline is potassium hydroxide.

8. The process of claim 1 wherein the purified product of step C subsequently undergoes crystallization, evaporation, or drying.

9. The process of claim 1 wherein the temperature of step B is between about 130° C. and about 140° C.

10. The process of claim 1 wherein the duration of time of step B is between about 3 and about 4 hours.

11. A purified lactic acid prepared according to the process of claim 1.

12. The purified lactic acid of claim 11 wherein the lactic salt is potassium lactate.

13. The purified lactic acid of claim 11 wherein the lactic salt has a concentration greater than 75%.

14. The purified lactic acid of claim 11 wherein the alkaline is selected from the group consisting of an alkaline oxide, an alkaline hydroxide, an alkaline carbonate, an alkaline bicarbonate, and an alkaline earth metal.

15. The purified lactic acid of claim 11 wherein the alkaline is potassium hydroxide.

16. The purified lactic acid of claim 11 wherein the temperature of step B of the process is between about 130° C. and about 140° C.

17. The purified lactic acid of claim 11 wherein the duration of time of step B of the process is between about 3 and about 4 hours.

18. The purified lactic acid of claim 11 wherein the lactic acid has a coloration lower than 50 Hazen.

19. The purified lactic acid of claim 11 wherein the lactic acid has a neutral odor.

20. A process for producing and purifying a mono or di-valent lactic salt of a concentration higher than 60% characterised in that it consists of the steps of:
reacting the molten lactide with a compound of the oxide, hydroxide, carbonate or bicarbonate of an alkaline, or alkaline earth metal, optionally in the presence of a suitable catalyst;
promoting the growth of odorant or odor precursor compounds by subjecting the solution to a heat treatment at a temperature between about 115 and 160° C. and for a duration of about 2 hours to about 6 hours; and
purifying the product obtained at the previous step in order to remove the compounds formed.
